(19) United States
(12) Patent Application Publication
(10) Pub. No.: US 2018/0318028 A1
(43) Pub. Date: Nov. 8, 2018

(54) MEDICAL DEVICE CARTON WITH SELF-LOCKING TAB AND METHOD

(71) Applicant: Medtronic Vascular, Inc., Santa Rosa, CA (US)

(72) Inventors: Courtney Spens, Santa Rosa, CA (US); Jeffrey Barnell, Santa Rosa, CA (US)

(21) Appl. No.: 15/585,533

(22) Filed: May 3, 2017

(51) Int. Cl. A61B 50/30; B65B 5/04; B65B 7/16; B65D 5/02; B65D 77/02

(52) U.S. Cl. CPC A61B 50/30; B65B 5/04; A61B 2050/005; B65D 5/0254; B65D 77/02; B65B 7/16

(57) ABSTRACT

A carton for a medical device includes a cover and an inner sidewall coupled to the cover. The inner sidewall includes a locking tab receiving slot. The carton further includes a first outer sidewall assembly comprising a self-locking tab configured to be inserted into the locking tab receiving slot, the self-locking tab having a first width greater than a second width of the locking tab receiving slot. The self-locking tab utilizes the geometry of the carton to create the seal. The self-locking tab is cut in a specific shape such that when it is inserted into the locking tab receiving slot, the self-locking tab locks the carton closed. Further, a perforated cut around the locking tab receiving slot allows a locking tab release flap of the carton to be lifted, freeing the self-locking tab so the carton may be opened.

### BACKGROUND

### Field

The present application relates to packaging for a medical device and method.

### Description of the Related Art

Aortic stent graft delivery systems are packaged in a carton with an end-seal. The end-seal is an adhesive sticker with a tearable strip which is place on the end of the carton so the closure flap of the carton cannot be removed. When the delivery system is ready for use, the tearable strip is removed and the closure flap is opened.

Problems occur when the end-seal inadvertently lifts from the carton, e.g., during storage, shipping, or other handling of the carton. The lifted end-seal gives the end use the impression the carton has been previously opened or handled in a manner that might suggest the delivery system is damaged. This often results in the carton being returned and rebuilt.

### SUMMARY

In accordance with one embodiment, a carton for a medical device includes a cover and an inner sidewall coupled to the cover. The inner sidewall includes a locking tab receiving slot. The carton further includes a first outer sidewall assembly comprising a self-locking tab configured to be inserted into the locking tab receiving slot, the self-locking tab having a first width greater than a second width of the locking tab receiving slot. The self-locking tab utilizes the geometry of the carton to create the seal. The self-locking tab is cut in a specific shape such that when it is inserted into the locking tab receiving slot, the self-locking tab locks the carton closed. Further, a perforated cut around the locking tab receiving slot allows a locking tab release flap of the carton to be lifted, freeing the self-locking tab so the carton may be opened.

The carton is sealed without use of adhesive end-seals. Accordingly, problems associated with lifted end-seals are avoided. For example, from a cost perspective, the self-locking tab eliminates the cost and labor associated with affixing end-seals. The self-locking tab eliminates the significant cost of rebuilding cartons due to faulty end seals. Further, by keeping the carton and medical device contained therein in the field, a better shelf life and inventory utilization is achieved.

### BRIEF DESCRIPTION OF DRAWINGS

FIG. 1 is a perspective view of a medical device assembly in accordance with one embodiment.

FIG. 2 is an enlarged perspective view of a region II of the medical device assembly of FIG. 1 in accordance with one embodiment.

FIG. 3 is a perspective view of a self-locking tab being inserted into a locking tab receiving slot of a carton of the medical device assembly of FIGS. 1 and 2 in accordance with one embodiment.

FIG. 4 is a top cross-sectional view of the self-locking tab locked within the locking tab receiving slot of the carton of the medical device assembly of FIG. 1 in accordance with one embodiment.

FIG. 5 is a perspective view of the carton of the medical device assembly of FIG. 1 in a locked state in accordance with one embodiment.

FIG. 6 is an enlarged top perspective view of the region VI of FIG. 5 in accordance with one embodiment.

FIG. 7 is a perspective view of the carton of the medical device assembly of FIG. 1 during opening in accordance with one embodiment.

FIG. 8 is an enlarged top perspective view of the region VIII of FIG. 7 in accordance with one embodiment.

FIG. 9 is a perspective view of the carton of FIG. 7 at a further stage during opening in accordance with one embodiment.

FIG. 10 is an enlarged top perspective view of the region X of FIG. 9 in accordance with one embodiment.

FIG. 11 is a laid-out plan view of the carton of the medical device assembly of FIG. 1 in accordance with one embodiment.

Common reference numerals are used throughout the drawings and detailed description to indicate like elements.

### DETAILED DESCRIPTION

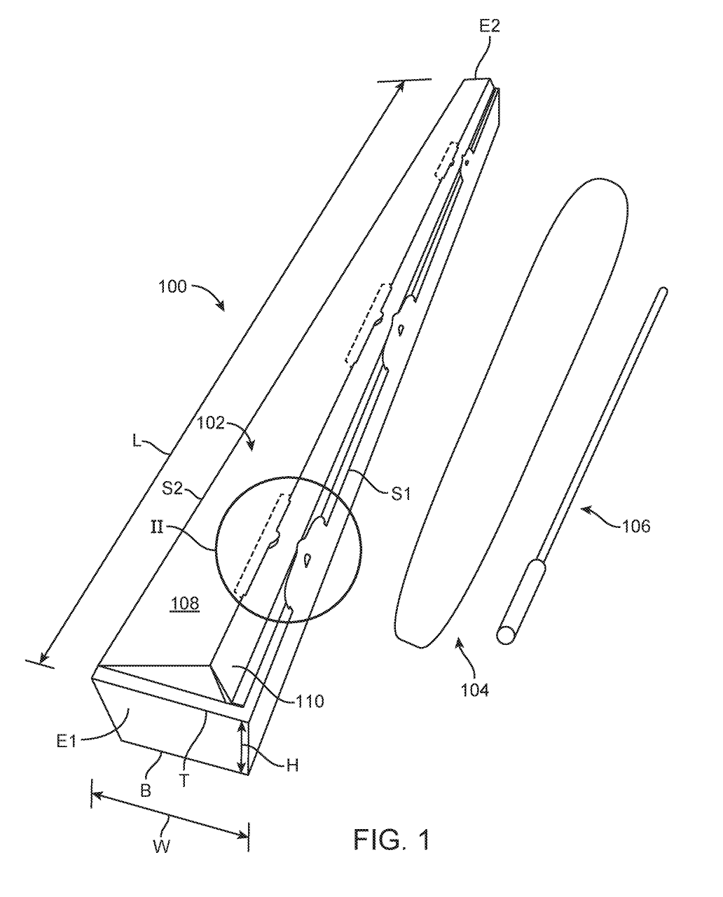

FIG. 1 is a perspective view of a medical device assembly 100 in accordance with one embodiment. Medical device assembly 100 includes a carton 102, a pouch 104, and a medical device 106. In one embodiment, medical device 106 is an endoluminal delivery system for delivering one or more prosthesis, e.g., stent-grafts, to a patient. Although one example of medical device 106 is provided, in other embodiments, medical device 106 is any one of a number of medical devices.

Medical device 106 is contained in a sterile condition within pouch 104 prior to use, e.g., during shipping and storage. Pouch 104 forms a sterile barrier around medical device 106. Pouch 104 can include one or more individual pouches.

Medical device 106 is contained within pouch 104, which is contained within carton 102. Carton 102, sometimes called a box, protects medical device 106 including pouch 104 prior to use. For example, medical device 106 including pouch 104 are contained within carton 102 during shipping, storage, and generally prior to use. Carton 102 can include one or more labels to identify the specific medical device 106 contained therein as well as other relevant information.

Carton 102 is illustrated in a semi-closed position in FIG. 1. For example, medical device 106 is contained within pouch 104. Pouch 104 including medical device 106 are placed within carton 102. After being placed within carton 102, a cover 108 is closed to seal pouch 104 including medical device 106 within carton 102. Cover 108 is illustrated in a partially closed state in FIG. 1.

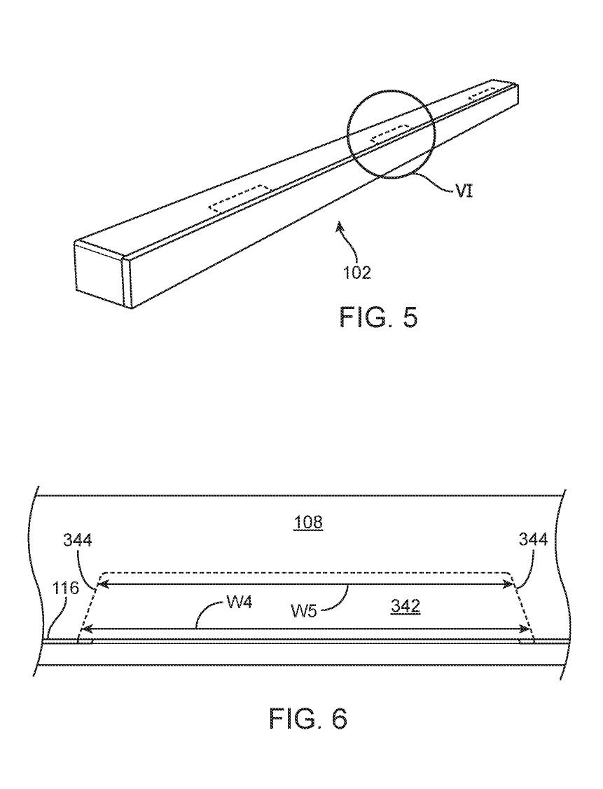

In its closed state (see FIG. 5 for example), carton 102 is rectangular having a length L, a width W, and a height H. In this embodiment, carton 102 is a long narrow box having length L much greater than width W and height H. For clarity, a direction parallel to length L is referred to as a length direction, a direction parallel to width W is referred to as the width direction, and a direction parallel to height H is referred to as the height direction. In accordance with this embodiment, carton 102 is rectangular and includes a base B, an opposite top T, a first sidewall S1, and opposite second sidewall S2, a first end El, and an opposite second end E2.

Although a particular shaped carton 102 is described and illustrated, carton 102 can take any one of a number of shapes depending upon the particular medical device 106 to be contained therein. Carton 102 is fabricated of a rigid sturdy material typically used for packaging such as corrugated cardboard, plastic, thin fibrous plastic, or other packaging material.

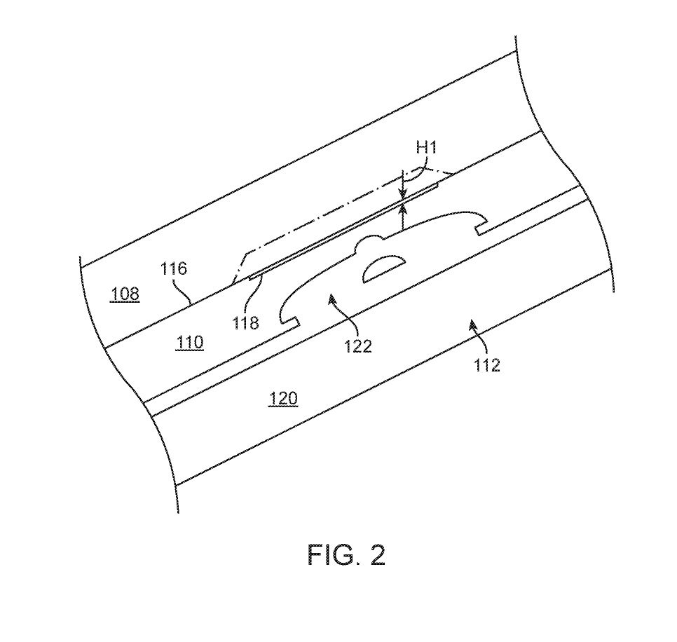

FIG. 2 is an enlarged perspective view of a region II of medical device assembly 100 of FIG. 1 in accordance with one embodiment. Referring now to FIGS. 1 and 2 together, carton 102 includes cover 108, an inner sidewall 110, and a first outer sidewall assembly 112. Inner sidewall 110 is coupled to cover 108 along a cover sidewall edge 116, i.e., inner sidewall 110 is folded from cover 108 along cover sidewall edge 116. Cover sidewall edge 116 is sometime called a cover sidewall fold 116. Cover 108 and inner sidewall 110 are sometimes referred to as a closure flap in accordance with one embodiment.

Carton 102 is assembled such that an enclosure into which pouch 104 and medical device 106 are inserted is defined by base B, first sidewall 51, second sidewall S2, first end El, and second end E2. Inner sidewall 110 is then inserted into the enclosure and adjacent first sidewall S1 until cover 108 is parallel to base B. Cover 108 forms top T.

A locking tab receiving slot 118, i.e., an opening, is formed within inner sidewall 110 and along cover sidewall edge 116. Locking tab receiving slot 118 is a long narrow opening extending in the length direction of carton 102. First outer sidewall assembly 112 includes a first outer sidewall 120 and a self-locking tab 122 extending from the first outer sidewall 120. Locking tab receiving slot 118 has a height H1 in the height direction approximately equal to the thickness of the material of carton 102 to allow self-locking tab 122 to be inserted therein.

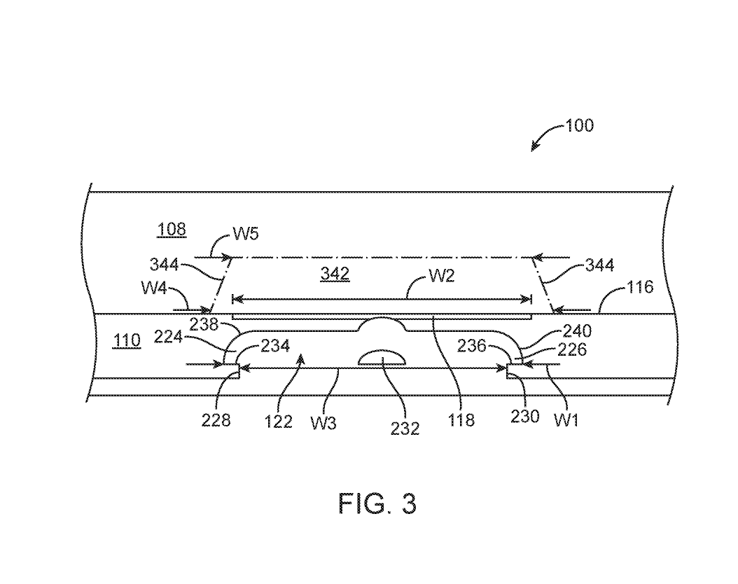

FIG. 3 is a perspective view of self-locking tab 122 being inserted into locking tab receiving slot 118 of carton 102 of medical device assembly 100 of FIGS. 1 and 2 in accordance with one embodiment. Referring now to FIGS. 1, 2, and 3 together, self-locking tab 122 includes wings 224, 226, inner sidewall engaging grooves 228, 230, and a tab grasping aperture 232.

Wings 224, 226 are opposite one another and protrude in the length direction. Wings 224, 226 define a first width W1 of self-locking tab 122, i.e., the furthermost distance between wings 224, 226. First width W1 of self-locking tab 122 is greater than a second width W2 of locking tab receiving slot 118. First width W1 and second width W2 are in the length direction. Wings 224, 226 include inner sidewall engaging lips 234, 236 that extend in the length direction. Wings 224, 226 further include curve shoulders 238, 240 extending from lips 234, 236, respectively. Curved shoulders 238, 240 taper inwards from lips 234, 236 facilitating insertion of self-locking tab 122 into locking tab receiving slot 118 as discussed further below. In one embodiment, self-locking tab 122 is shaped as a trapezoid.

Inner sidewall engaging grooves 228, 230 are narrow grooves configured to receive inner sidewall 110 therein. A third width W3 of self-locking tab 122 between inner sidewall engaging grooves 228, 230, i.e., the distance between inner sidewall engaging grooves 228, 230, is less than or equal to second width W2 of locking tab receiving slot 118.

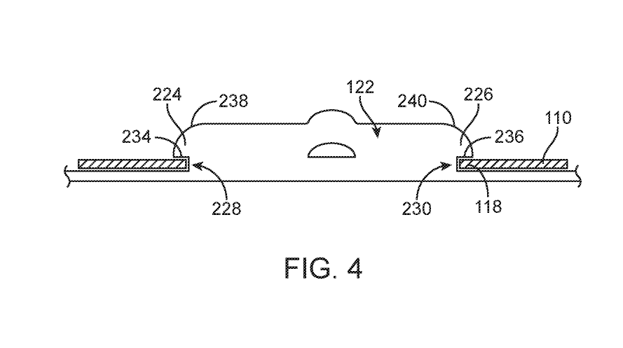

FIG. 4 is a top cross-sectional view of self-locking tab 122 locked within locking tab receiving slot 118 of carton 102 of medical device assembly 100 of FIG. 1 in accordance with one embodiment. In FIG. 4, only a cutaway view of inner sidewall 110 and self-locking tab 122 are illustrated for simplicity.

Referring now to FIGS. 3 and 4 together, to seal carton 102, self-locking tab 122 is inserted into locking tab receiving slot 118. Self-locking tab 122 is inserted into locking tab receiving slot 118 such that shoulders 238, 240 slide along inner sidewall 110 and compresses wings 224, 226 and generally self-locking tab 122 as self-locking tab 122 passes through locking tab receiving slot 118. As wings 224, 226 pass-through locking tab receiving slot 118, wings 224, 226 self-expand such that inner sidewall 110 is received within inner sidewall engaging grooves 228, 230 and locked therein as illustrated in FIG. 4. Further, as wings 224, 226 pass-through locking tab receiving slot 118, wings 224, 226 self-expand such that inner sidewall engaging lips 234, 236 engage inner sidewall 110 as also illustrated in FIG. 4. Generally, self-locking tab 122 is inserted into locking tab receiving slot 118 and self-locks therein preventing self-locking tab 122 from being pulled back out after insertion.

FIG. 5 is a perspective view of carton 102 of medical device assembly 100 of FIG. 1 in a locked state in accordance with one embodiment. FIG. 6 is an enlarged top perspective view of the region VI of FIG. 5 in accordance with one embodiment. As illustrated in FIGS. 5 and 6, self-locking tab 122 is fully inserted within locking tab receiving slot 118 thus self-sealing carton 102.

As set forth above, self-locking tab 122 utilizes the geometry of carton 102 to create the seal. Self-locking tab 122 is cut in a specific shape such that when it is inserted into locking tab receiving slot 118, cut into carton 102, self-locking tab 122 locks carton 102 closed.

Carton 102 is sealed without use of adhesive end-seals. Accordingly, problems associated with lifted end-seals are avoided. For example, from a cost perspective, self-locking tab 122 eliminates the cost and labor associated with affixing end-seals. Self-locking tab 122 eliminates the significant cost of rebuilding cartons due to faulty end seals. Further, by keeping medical device assembly 100 in the field, a better shelf life and inventory utilization is achieved.

Referring now to FIGS. 3, 5, and 6 together, to facilitate opening of carton 102 for removal of pouch 104 including medical device 106, carton 102 further includes a locking tab release flap 342. In this embodiment, locking tab release flap 342 is integral with cover 108 and is defined by one or more breakaways 344. More particularly, breakaways 344 are perforated cuts between locking tab release flap 342 and cover 108. Breakaways 344 are configured to break upon application of force by the user desiring to remove medical device 106 from carton 102.

Locking tab release flap 342 extends from cover sidewall edge 116 including locking tab receiving slot 118 in the width direction. Locking tab release flap 342 has a fourth width W4 at cover sidewall edge 116 greater than first width W1 of self-locking tab 122. Generally, locking tab release flap 342 is greater in area than self-locking tab 122, e.g., mimics the shape of self-locking tab 122, thus freeing self-locking tab 122 upon removal of locking tab release flap 342.

In accordance with this embodiment, locking tab release flap 342 has a trapezoidal shape and tapers from fourth width W4 to a fifth width W5. Fifth width W5 is less than fourth width W4. The trapezoidal shape of locking tab release flap 342 ensures breakaways 344 tear evenly and consistently every time without ripping of carton 102. Although a trapezoidal shape of locking tab release flap 342 is illustrated, in other embodiments, locking tab release flap 342 has other shapes, e.g., is rectangular.

When carton 102 is in the closed and locked position as illustrated in FIGS. 5 and 6, locking tab release flap 342 abuts self-locking tab 122 thus keeping self-locking tab 122 in place.

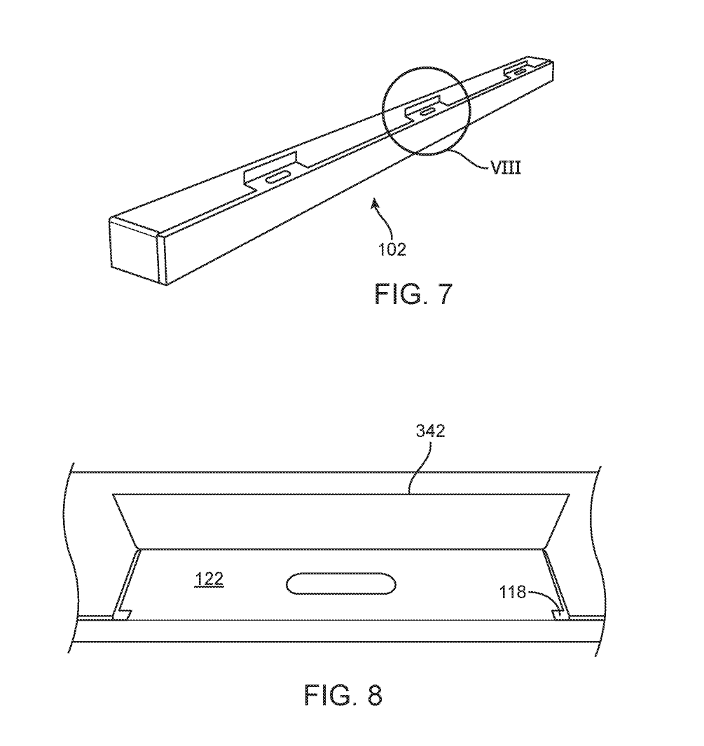

FIG. 7 is a perspective view of carton 102 of medical device assembly 100 of FIG. 1 during opening in accordance with one embodiment. FIG. 8 is an enlarged top perspective view of the region VIII of FIG. 7 in accordance with one embodiment.

Referring now to FIGS. 3, 7 and 8 together, locking tab release flap 342 is grasped by the user and pulled away from locking tab receiving slot 118. This application of force breaks breakaways 344 (see FIGS. 3, 6) opening locking tab release flap 342 and thus removing locking tab release flap 342 from self-locking tab 122. This frees self-locking tab 122 for removal and opening of carton 102. Generally, when medical device 106 is ready for use, breakaways 344, i.e., a perforated cut around locking tab receiving slot 118, allow locking tab release flap 342 to be lifted, freeing self-locking tab 122 so carton 102 may be opened.

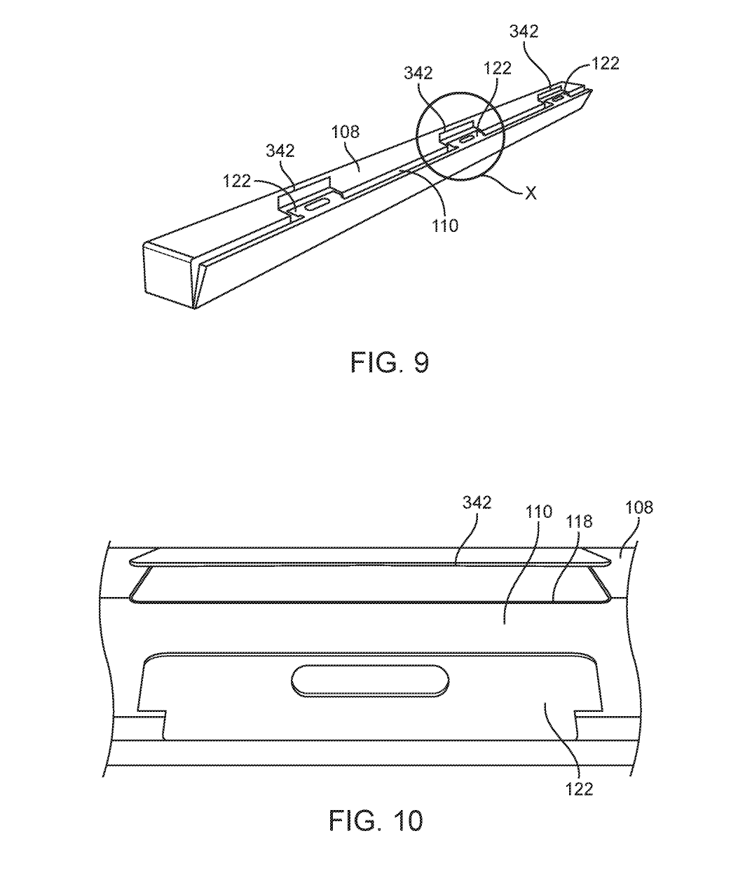

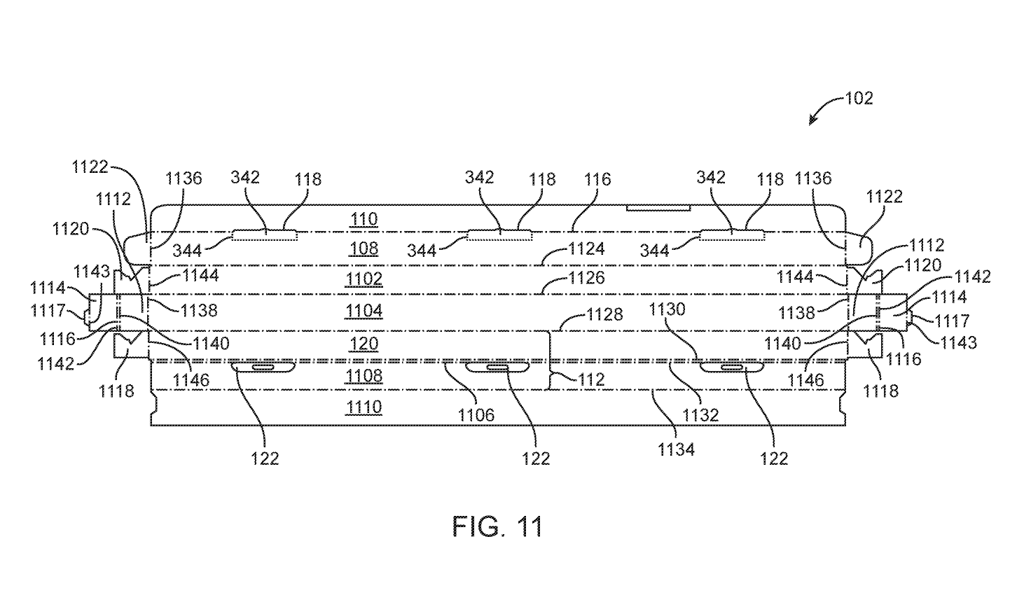

FIG. 9 is a perspective view of carton 102 of FIG. 7 at a further stage during opening in accordance with one embodiment. FIG. 10 is an enlarged top perspective view of the region X of FIG. 9 in accordance with one embodiment. Referring now to FIGS. 9 and 10 together, self-locking tab 122 is removed from cover 108 and inner sidewall 110 thus opening carton 102. Although a single self-locking tab 122 and associated locking tab receiving slot 118/locking tab release flap 342 are discussed above, generally, carton 102 includes at least one self-locking tab 122 and associated locking tab receiving slot 118/locking tab release flap 342. For example, in accordance with this specific embodiment, carton 102 includes three self-locking tabs 122 and associated locking tab receiving slots 118/locking tab release flaps 342.

Further, self-locking tab 122 and associated locking tab receiving slot 118/locking tab release flap 342 can be formed in other locations than those shown on carton 102. For example, self-locking tab 122 and associated locking tab receiving slot 118/locking tab release flap 342 can be formed at a corner. In another example, self-locking tab 122 is form at the end of a strap that extends at least partially around the carton and is inserted into an associated locking tab receiving slot 118.

FIG. 11 is a laid-out plan view of carton 102 of medical device assembly 100 of FIG. 1 in accordance with one embodiment. In FIG. 11, a solid line is a cut line, a dash dot line is a fold line, and a dotted line is a perforated cut line, also called a breakaway.

Various features of carton 102 as illustrated in FIG. 11 are as follows: inner sidewall 110, cover 108, a second outer sidewall 1102, an outer base 1104, first outer sidewall 120, a first outer sidewall connector 1106, a middle sidewall 1108, an inner base 1110, self-locking tabs 122, locking tab release flaps 342, outer end walls 1112, middle end walls 1114, end wall connectors 1116, tabs 1117, first outer sidewall end flaps 1118, second outer sidewall end flaps 1120, and inner end walls 1122.

Inner sidewall 110 is coupled to cover 108 by cover sidewall edge 116, also called a fold. Cover 108 is coupled to second outer sidewall 1102 by a fold 1124. Second outer sidewall 1102 is coupled to outer base 1104 by a fold 1126. Outer base 1104 is coupled to first outer sidewall 120 by a fold 1128. First outer sidewall 120 is coupled to first outer sidewall connector 1106 by a fold 1130. First outer sidewall connector 1106 is coupled to middle sidewall 1108 by a fold 1132. Middle sidewall 1108 is coupled to inner base 1110 by a fold 1134. Note that folds 116, 1124, 1126, 1128, 1130, 1132, 1134 extend in the length direction in the constructed and closed carton 102.

Inner end walls 1122 are coupled to the opposite ends of cover 108 by folds 1136. Outer base 1104 is coupled on the opposite ends to outer end walls 1112 by folds 1138. Outer end walls 1112 are coupled to end wall connectors 1116 by folds 1140. End wall connectors 1116 are coupled to middle end walls 1114 by folds 1142. Middle end walls 1114 are coupled to tabs 1117 by folds 1143. Note that folds 1136, 1138, 1140, 1142, 1143 extend in the width direction in the constructed and closed carton 102.

Second outer sidewall end flaps 1120 are coupled to the opposite ends of second outer sidewall 1102 by folds 1144. First outer sidewall end flaps 1118 are coupled to the opposite ends of first outer sidewall 120 by folds 1146. Note that folds 1144, 1146 extend in the height direction in the constructed and closed carton 102.

This disclosure provides exemplary embodiments. The scope is not limited by these exemplary embodiments. Numerous variations, whether explicitly provided for by the specification or implied by the specification or not, such as variations in structure, dimension, type of material and manufacturing process may be implemented by one of skill in the art in view of this disclosure.

What is claimed is:

1. A carton for a medical device comprising:
a cover;
an inner sidewall coupled to the cover, wherein the inner sidewall comprises a locking tab receiving slot; and
a first outer sidewall assembly comprising a self-locking tab configured to be inserted into the locking tab receiving slot, the self-locking tab having a first width greater than a second width of the locking tab receiving slot.

2. The carton of claim 1 further comprising a locking tab release flap coupled to the cover and extending from the locking tab receiving slot.

3. The carton of claim 2 wherein the locking tab release flap is coupled to the cover by a breakaway.

4. The carton of claim 3 wherein the breakaway comprises a perforated cut between the cover and the locking tab release flap.

5. The carton of claim 2 wherein the locking tab release flap has a fourth width greater than the first width of the self-locking tab.

6. The carton of claim 5 wherein the locking tab release flap has the fourth width at the locking tab receiving slot.

7. The carton of claim 6 wherein the locking tab release flap tapers from the fourth width to a fifth width less than the fourth width.

8. The carton of claim 7 wherein the locking tab release flap has a trapezoidal shape.

9. The carton of claim 1 wherein the self-locking tab comprises wings configured to be compressed when passing through the locking tab receiving slot.

10. The carton of claim 9 wherein the self-locking tab comprises inner sidewall engaging grooves configured to receive the inner sidewall therein.

11. The carton of claim 1 wherein the self-locking tab comprises a tab grasping aperture therein.

12. A medical device assembly comprising:
a medical device; and
a carton comprising:
a locking tab receiving slot; and
a first outer sidewall assembly comprising a self-locking tab inserted into the locking tab receiving slot to seal the carton around the medical device.

13. The medical device assembly of claim 12 wherein the medical device comprises an endoluminal delivery system.

14. The medical device assembly of claim 13 further comprising a sterile pouch around the endoluminal delivery system and within the carton.

15. The medical device assembly of claim 12 wherein the carton further comprises a locking tab release flap extending from the locking tab receiving slot.

16. The medical device assembly of claim 12 wherein the carton further comprises:
a cover; and
an inner sidewall coupled to the cover, wherein the inner sidewall comprises the locking tab receiving slot.

17. The medical device assembly of claim 12 wherein the self-locking tab has a first width greater than a second width of the locking tab receiving slot.

18. A method comprising:
inserting a medical device into a carton; and
inserting a self-locking tab into a locking tab receiving slot to seal the carton around the medical device, wherein the inserting comprises compressing the self-locking tab as the self-locking tab passes through the locking tab receiving slot.

19. The method of claim 18 wherein the self-locking tab expands upon passing through the locking tab receiving slot to seal the carton.

20. The method of claim 18 further comprising tearing a locking tab release flap from a cover to release the self-locking tab.
